(19) United States
(12) Patent Application Publication
(10) Pub. No.: US 2025/0095867 A1
(43) Pub. Date: Mar. 20, 2025

(54) METHODS AND SYSTEMS FOR PREDICTING STABILITY

(71) Applicant: AMGEN INC., Thousand Oaks, CA (US)

(72) Inventors: Elif Seyma BAYRAK, Westlake Village, CA (US); Cenk UNDEY, Redwood City, CA (US); Saleh ALKHALIFA, Malden, MA (US); Sriramvignesh MANI, Westborough, MA (US); Gerd R. KLEEMANN, Newbury Park, CA (US); Andrew C. LENNARD, Buckinghamshire (GB); Barbara RELLAHAN, Kensington, MD (US); Camilla A. SANTOS, Providence, RI (US)

(21) Appl. No.: 18/889,204

(22) Filed: Sep. 18, 2024

Related U.S. Application Data
(60) Provisional application No. 63/539,207, filed on Sep. 19, 2023.

(51) Int. Cl. G16H 70/40; G06N 20/00

(52) U.S. Cl. CPC G16H 70/40; G06N 20/00

(57) ABSTRACT

A computer-implemented method for determining stability of one or more target pharmaceutical materials, the method comprising: obtaining, via one or more processors, training characteristic data associated with one or more training pharmaceutical materials at one or more training timepoints, wherein the one or more training timepoints comprise at least a first predetermined timepoint; obtaining, via the one or more processors, target characteristic data associated with the one or more target pharmaceutical materials at one or more target timepoints, wherein the one or more target timepoints are earlier than the first predetermined timepoint; generating, via the one or more processors, one or more clusters of the one or more training pharmaceutical materials and the one or more target pharmaceutical materials based on the training characteristic data and the target characteristic data; selecting, via the one or more processors, a subset of the one or more training pharmaceutical materials based on the one or more clusters; training, via the one or more processors, a first computational model using the training characteristic data associated with the subset of the one or more training pharmaceutical materials at the one or more training timepoints; and determining, via the one or more processors, the stability of the one or more target pharmaceutical materials at the first predetermined timepoint based on the target characteristic data associated with the one or more target pharmaceutical materials using the trained first computational model.

### CROSS REFERENCE TO RELATED APPLICATIONS

The present application claims the benefit of priority under 35 U.S.C. § 119 (c) of U.S. Provisional Patent Application Ser. No. 63/539,207 filed on Sep. 19, 2023, and entitled “METHODS AND SYSTEMS FOR PREDICTING STABILITY,” which is herein incorporated by reference in its entirety.

### BACKGROUND

Stability of a pharmaceutical material describes the ability of the pharmaceutical material to retain its chemical, physical, microbiological and biopharmaceutical properties within specified conditions throughout its shelf-life. Stability affects the safety and efficacy of pharmaceutical materials. Degradation and impurities may cause a loss of efficacy and generate possible adverse effects. Therefore, understanding the stability of pharmaceutical materials over time is essential to ensure their quality and safety.

### SUMMARY

In one aspect, a computer-implemented method for determining stability of one or more target pharmaceutical materials comprises (a) obtaining, via one or more processors, training characteristic data associated with one or more training pharmaceutical materials at one or more training timepoints, wherein the one or more training timepoints comprise at least a first predetermined timepoint; (b) obtaining, via the one or more processors, target characteristic data associated with the one or more target pharmaceutical materials at one or more target timepoints, wherein the one or more target timepoints are earlier than the first predetermined timepoint; (c) generating, via the one or more processors, one or more clusters of the one or more training pharmaceutical materials and the one or more target pharmaceutical materials based on the training characteristic data and the target characteristic data; (d) selecting, via the one or more processors, a subset of the one or more training pharmaceutical materials based on the one or more clusters; (e) training, via the one or more processors, a first computational model using the training characteristic data associated with the subset of the one or more training pharmaceutical materials at the one or more training timepoints; and (f) determining, via the one or more processors, the stability of the one or more target pharmaceutical materials at the first predetermined timepoint based on the target characteristic data associated with the one or more target pharmaceutical materials using the trained first computational model.

In some embodiments, the training characteristic data comprise one or more training characteristic categories. In some embodiments, the target characteristic data comprise one or more target characteristic categories. In some embodiments, the one or more training characteristic categories comprise at least one of a high molecular weight value (e.g., a high molecular percentage), a concentration, or a pH value. In some embodiments, the one or more target characteristic categories comprise at least one of a high molecular weight value, a concentration, or a pH value.

In some embodiments, generating the one or more clusters of the one or more training pharmaceutical materials and the one or more target pharmaceutical materials comprises generating the one or more clusters using at least one of a dimensionality reduction technique or a clustering technique. In some embodiments, the method further comprises ranking the one or more training characteristic categories based on a level of impact on the determined stability. In some embodiments, the one or more training pharmaceutical materials are different from the one or more target pharmaceutical materials. In some embodiments, the one or more training timepoints further comprise a second predetermined timepoint, and the first predetermined timepoint is earlier than the second predetermined timepoint.

In some embodiments, the method further comprises determining the stability of the one or more target pharmaceutical materials at the second predetermined timepoint based on the target characteristic data associated with the one or more target pharmaceutical materials using the trained first computational model. In some embodiments, the method further comprises causing a display to present a visual indication of the determined stability. In some embodiments, the method further comprises generating one or more recommendations to adjust at least one storage status associated with the one or more target pharmaceutical materials based on the determined stability. In some embodiments, the method comprises adjusting, via the one or more processors, at least one storage status associated with the one or more target pharmaceutical materials based on the determined stability and/or the one or more recommendations. In some embodiments, the method further comprises training a second computational model using the training characteristic data associated with the one or more training pharmaceutical materials at the one or more training timepoints; comparing the trained first computational model and the trained second computational model based on a level of prediction accuracy; and determining the stability of the one or more target pharmaceutical materials at the first predetermined timepoint based on the comparison between the trained first computational model and the trained second computational model and the target characteristic data associated with the one or more target pharmaceutical materials.

In another aspect, a computer system for determining stability of one or more target pharmaceutical materials comprises a memory storing instructions; and one or more processors configured to execute the instructions to perform operations including: (a) obtaining training characteristic data associated with one or more training pharmaceutical materials at one or more training timepoints, wherein the one or more training timepoints comprise at least a first predetermined timepoint; (b) obtaining target characteristic data associated with the one or more target pharmaceutical materials at one or more target timepoints, wherein the one or more target timepoints are earlier than the first predetermined timepoint; (c) generating one or more clusters of the one or more training pharmaceutical materials and the one or more target pharmaceutical materials based on the training characteristic data and the target characteristic data; (d) selecting a subset of the one or more training pharmaceutical materials based on the one or more clusters; (e) training a first computational model using the training characteristic data associated with the subset of the one or more training pharmaceutical materials at the one or more training timepoints; and (f) determining the stability of the one or more target pharmaceutical materials at the first predetermined timepoint based on the target characteristic data associated with the one or more target pharmaceutical materials using the trained first computational model.

In another aspect, a non-transitory computer-readable medium contains instructions for determining stability of one or more target pharmaceutical materials that, when executed by a processor, cause the processor to perform a method comprising: (a) obtaining training characteristic data associated with one or more training pharmaceutical materials at one or more training timepoints, wherein the one or more training timepoints comprise at least a first predetermined timepoint; (b) obtaining target characteristic data associated with the one or more target pharmaceutical materials at one or more target timepoints, wherein the one or more target timepoints are earlier than the first predetermined timepoint; (c) generating one or more clusters of the one or more training pharmaceutical materials and the one or more target pharmaceutical materials based on the training characteristic data and the target characteristic data; (d) selecting a subset of the one or more training pharmaceutical materials based on the one or more clusters; (c) training a first computational model using the training characteristic data associated with the subset of the one or more training pharmaceutical materials at the one or more training timepoints; and (f) determining the stability of the one or more target pharmaceutical materials at the first predetermined timepoint based on the target characteristic data associated with the one or more target pharmaceutical materials using the trained first computational model.

In yet another aspect, a computer-implemented method for determining stability of a target pharmaceutical material, the method comprising: (a) obtaining, via one or more processors, training characteristic data associated with one or more training pharmaceutical materials at one or more training timepoints, wherein the one or more training timepoints comprise at least a first predetermined timepoint; (b) obtaining, via the one or more processors, target characteristic data associated with the target pharmaceutical material at one or more target timepoints, wherein the one or more target timepoints are earlier than the first predetermined timepoint; (c) generating, via the one or more processors, one or more clusters of the one or more training pharmaceutical materials and the target pharmaceutical material based on the training characteristic data and the target characteristic data; (d) selecting, via the one or more processors, a subset of the one or more training pharmaceutical materials based on the one or more clusters; (c) training, via the one or more processors, a first computational model using the training characteristic data associated with the subset of the one or more training pharmaceutical materials at the one or more training timepoints; and (f) determining, via the one or more processors, the stability of the target pharmaceutical material at the first predetermined timepoint based on the target characteristic data associated with the target pharmaceutical material using the trained first computational model.

### BRIEF DESCRIPTION OF DRAWINGS

The skilled artisan will understand that the figures, described herein, are included for purposes of illustration and are not limiting on the present disclosure. The drawings are not necessarily to scale, emphasis instead being placed upon illustrating the principles of the present disclosure. It is to be understood that, in some instances, various aspects of the described implementations may be shown exaggerated or enlarged to facilitate an understanding of the described implementations. In the drawings, like reference characters throughout the various drawings generally refer to functionally similar and/or structurally similar components.

FIG. 1 is a block diagram of an exemplary system 100 for determining stability of one or more target pharmaceutical materials, in accordance with some embodiments of the technology described herein.

FIG. 2 is a flowchart of an exemplary process 200 for determining stability of one or more target pharmaceutical materials, in accordance with some embodiments of the technology described herein.

FIG. 3 is an exemplary flow of data selection, in accordance with some embodiments of the technology described herein.

FIG. 4 depicts an exemplary table 402 showing characteristic data of one or more pharmaceutical materials, an exemplary plot 404 showing stability indicating attribute profiles of one or more pharmaceutical materials, and an exemplary plot 406 showing the number of lots (e.g., samples) of one or more pharmaceutical materials, in accordance with some embodiments of the technology described herein.

FIG. 5 is an exemplary table showing correlations between one or more characteristic categories and HMW values, in accordance with some embodiments of the technology described herein.

FIG. 6 depicts exemplary methods used for assessment of similarity among different pharmaceutical materials, in accordance with some embodiments of the technology described herein.

FIG. 7 depicts exemplary results from one clustering technique, in accordance with some embodiments of the technology described herein.

FIG. 8 depicts an exemplary method for predicting stability of one or more target pharmaceutical materials, in accordance with some embodiments of the technology described herein.

FIG. 9 depicts exemplary diagrams depicting the impact of each characteristic category on stability predictions, in accordance with some embodiments of the technology described herein.

FIG. 10 depicts other exemplary diagrams depicting the impact of each characteristic category on stability predictions, in accordance with some embodiments of the technology described herein.

FIG. 11 depicts two exemplary plots showing comparison between the predicted values and measured values for a stability indicating attribute, in accordance with some embodiments of the technology described herein.

FIG. 12 is a schematic diagram of an illustrative computing device with which aspects described herein may be implemented.

### DETAILED DESCRIPTION

The inventors have developed computational models (e.g., machine learning techniques) for predicting stability for different pharmaceutical materials (e.g., training pharmaceutical materials, target pharmaceutical materials). Conventional methods of predicting stability for pharmaceutical materials for longer periods often involve years (e.g., 24 months) of real-time wet lab experimental data in order to predict long-term stability (e.g., 36 months), which is both time-consuming and resource-intensive. Additionally, real-time stability testing involves storing the drug under specific conditions for an extended period, with periodic analyses to assess any changes in its chemical composition or physical properties. Although these approaches provide insights into the pharmaceutical material's shelf life, the lengthy experimental timelines make it challenging to expedite the drug development process, leading to delays in bringing new treatments to market.

The systems and methods disclosed herein can overcome these challenges by predicting stability for different pharmaceutical materials in less time. The systems and methods disclosed herein can reduce the reliance on lengthy experimental timelines, enabling the prediction of long-term stability with less experimental data. Therefore, the systems and methods disclosed herein can accelerate the drug development process and allow for more efficient resource allocation, ultimately helping bring new treatments to market faster.

The systems and methods disclosed herein can comprise a machine learning model developed to predict values of one or more stability indicating attribute (e.g., the formation of High Molecular Weight (HMW) species) during pharmaceutical product storage under recommended storage conditions (RSC) for different pharmaceutical materials. The prediction can be used to support regulatory filings. The systems and methods disclosed herein can provide insights into product stability within weeks or months that traditionally can take years to generate. Thus, the systems and methods disclosed herein can accelerate processes such as molecule candidate selection, product development, and initiation of clinical trials, which are useful for launching the pharmaceutical material to market for earlier patient access. The systems and methods disclosed herein can predict the attainable shelf life for a pharmaceutical material and thereby, help in scrap (e.g., wastage of pharmaceutical materials) reduction, and evaluate product stability risk under unexpected storage conditions.

The systems and methods disclosed herein can use prior knowledge to build computational models (e.g., machine learning models) that predict the stability of pharmaceutical materials using one or more stability indicating attributes (e.g., the formation of HMW species after long-term storage under recommended storage conditions (RSC)) with higher accuracy than traditional methods that generally do not include prior knowledge. The systems and methods disclosed herein can learn the dynamics of the product stability profiles and provide accurate prediction considering early product stability profiles and potential product stability indicating attributes (e.g., HMW, fragmentation, changes in charge species, etc.).

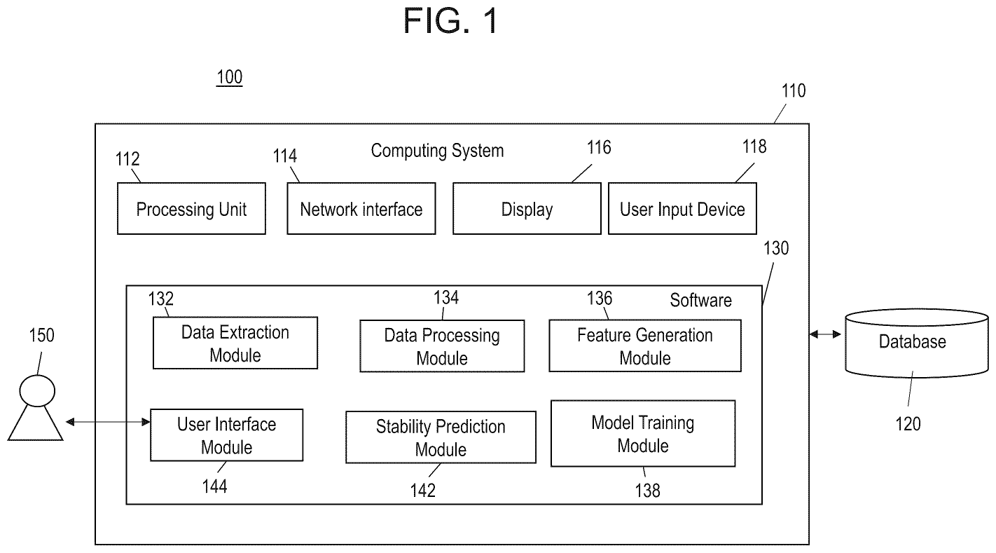

FIG. 1 is a block diagram of an exemplary system 100 for predicting stability of one or more target pharmaceutical materials, in accordance with some embodiments of the technology described herein.

System 100 includes a computing system 110 coupled to a database 120. Computing system 110 can be configured to have software 130 execute thereon to perform various functions in connection with predicting stability of one or more target pharmaceutical materials. Computing system 110 can comprise a single computing device or include multiple co-located and/or distributed computing devices communicatively coupled by one or more networks. The computing system 110 can comprise one or multiple computing devices of any suitable type. For example, the computing system 110 may be a portable computing device (e.g., laptop, a smartphone) or a fixed computing device (e.g., a desktop computer, a server). When computing system 110 includes multiple computing devices, the device(s) may be physically co-located (e.g., in a single room) or distributed across multiple physical locations. In some embodiments, the computing system 110 may be part of a cloud computing infrastructure.

In some embodiments, the computing system 110 may be operated by one or more user(s) 150 such as one or more researchers, health professionals, and/or other individual(s). For example, the user(s) 150 may provide characteristic data (e.g., training characteristic data, target characteristic data) associated with one or more pharmaceutical materials as input to the computing system 110 (e.g., by uploading one or more files), and/or may provide user input specifying processing or other methods to be performed on the characteristic data associated with one or more pharmaceutical materials (e.g., training pharmaceutical materials, target pharmaceutical materials).

In the example embodiment shown in FIG. 1, computing system 110 includes a processing unit 112, a network interface 114, a display 116, a user input device 118, and a software 130. Processing unit 112 includes one or more processors, each of which may be a programmable microprocessor that executes software instructions stored in memory to execute some or all of the functions of computing system 110 as described herein. Alternatively, one, some or all of the processors in processing unit 112 may be other types of processors (e.g., application-specific integrated circuits (ASICs), field-programmable gate arrays (FPGAs), etc.), and the functionality of computing system 110 as described herein may instead be implemented, in part or in whole, in hardware. Memory may include one or more physical memory devices or units containing volatile and/or non-volatile memory. Any suitable memory type or types may be used, such as read-only memory (ROM), solid-state drives (SSDs), hard disk drives (HDDs), and so on.

Network interface 114 may include any suitable hardware (e.g., front-end transmitter and receiver hardware), firmware, and/or software configured to communicate with external devices and/or systems (e.g., a client device, or one or more servers maintaining database 120) via one or more networks using one or more communication protocols. For example, network interface 114 may be or include an Ethernet interface, and/or include a wireless local area network (LAN) interface, etc.

Display 116 may use any suitable display technology (e.g., LED, OLED, LCD, etc.) to present information to a user 150, and user input device 118 may be a keyboard or other suitable input device. In some embodiments, display 116 and user input device 118 are integrated within a single device (e.g., a touchscreen display). Generally, display 116 and user input device 118 may combine to enable a user 150 to interact with user interfaces (e.g., graphical user interfaces (GUIs)) provided by computing system 110, such as those discussed in further detail below. In some embodiments, however, computing system 110 does not include display 116 and/or user input device 118, or one or both of display 116 and user input device 118 are included in another computer or system that is communicatively coupled to computing system 110 (e.g., a client device not shown in FIG. 1).

As shown in FIG. 1, software 130 includes multiple software modules for processing characteristic data (e.g., training characteristic data, target characteristic data) associated with one or more pharmaceutical materials (e.g., training pharmaceutical materials, target pharmaceutical materials), such as a data extraction module 132, a data processing module 134, a feature generation module 136, a model training module 138, and a stability prediction module 142. In the embodiment of FIG. 1, the software 130 additionally includes a user interface module 144 for obtaining user input.

In some embodiments, data extraction module 132 is generally responsible for retrieving/obtaining data (e.g., characteristic data associated with one or more pharmaceutical materials) from the database 120. In some embodiments, data extraction module 132 retrieves training characteristic data associated with one or more training pharmaceutical materials at one or more training timepoints based on user input detected by user interface module 144. In some embodiments, data extraction module 132 retrieves target characteristic data associated with one or more target pharmaceutical materials based on user input detected by user interface module 144. For example, user interface module 144 may generate and/or populate a GUI, and cause display 116 to present the GUI to a user. The user may then operate user input device 118 to enter one or more pharmaceutical materials via the GUI, and data extraction module 132 may retrieve characteristic data associated with the one or more pharmaceutical materials. In some embodiments, the database 120 includes raw data, and data extraction module 132 constructs similar data structure(s). For example, data extraction module 132 may generate data in a more readily usable form.

In some embodiments, data processing module 134 is generally responsible for processing the data (e.g., characteristic data associated with one or more pharmaceutical materials) from the database 120 extracted via the data extraction module 134. Data processing module 134 can clean, filter, and transform the data from the database 120 (e.g., raw data of training characteristic data associated with one or more training pharmaceutical materials) using any type of computational or mathematical techniques, including machine learning techniques. Data processing module 134 can process the data based on user input detected by user interface module 144. For example, user interface module 144 may generate and/or populate a GUI, and cause display 116 to present the GUI to a user. The user may then operate user input device 118 to enter one or more instructions related to processing the data via the GUI, and data processing module 134 may process the data based on the user input. In some embodiments, the database 120 includes raw data (e.g., data without any processing steps), and data processing module 134 can process the raw data so the data can be utilized by other modules (e.g., feature generation module 130) or processes. For example, data extraction module 132 may normalize the raw data, or may generate data in a more readily usable form (e.g., a table demonstrating the characteristic data of one or more pharmaceutical materials).

In some embodiments, the feature generation module 136 obtains processed data from the database 120, the data extraction module 132, and/or the data processing module 134, and uses the processed data to generate sets of features. Such features can be characteristic categories (e.g., training characteristic categories, target characteristic categories), including but not limited to, high molecular weight, concentration, and chain length. For example, the feature generation module 136 may generate a set of features for high molecular weight values of one pharmaceutical material at different timepoints. In some embodiments, the feature generation module 136 generates a set of features by including at least some of the obtained data in the set of features. For example, the feature generation module 136 may generate the set of features to include characteristic data associated with one or more pharmaceutical materials. For example, feature generation module 136 may generate the set of features to include a two-dimensional (2D) matrix that that stores values of high molecular weight, concentration, chain length as y dimension, and different timepoints as x dimension. The generated 2D matrices may be provided as input to the machine learning model. Additionally, or alternatively, the feature generation module 136 may generate a set of features including encoded data. For example, the characteristic data associated with one or more pharmaceutical materials may be one-hot encoded. The feature generation module 136 may include additional or alternative features in the set of features, as aspects of the technology described herein are not limited in this respect.

In some embodiments, the model training module 138 may be configured to train one or more models (e.g., machine learning models) to predict stability for one or more target pharmaceutical materials. In some embodiments, the model training module 138 trains a machine learning model using training characteristic data associated with one or more training pharmaceutical materials. For example, the model training module 138 may obtain training characteristic data from the database 120. In some embodiments, the model training module 138 may provide trained machine learning model(s) to the database 120. Techniques for training a machine learning model are described elsewhere herein.

In some embodiments, the stability prediction module 142 obtains one or more sets of features from the feature generation module 136, obtains a trained machine learning model from the model training module 138 and database 120 (which may be a data store of any suitable type), and processes the obtained set(s) of features using the obtained machine learning model to obtain stability of the one or more target pharmaceutical materials. For example, the stability prediction module 136 may process the set of features generated using the trained machine learning model to obtain values of one or more stability indicating attributes for the one or more target pharmaceutical materials. Techniques for predicting stability using machine learning are described elsewhere herein. The stability indicating attributes may indicate values of high molecular weight of one pharmaceutical material in 2 years or 3 years. In some embodiments, the predicted stability may be output by the stability prediction module 142. For example, the predicted stability may be output to user(s) 150 via user interface module 144. Additionally, or alternatively, the predicted stability may be stored in memory and/or transmitted to one or more other computing devices and/or systems.

As shown in FIG. 1, system 100 also includes database 120. The database 120 may store model data, characteristic data associated with one or more pharmaceutical materials, and/or raw data associated with one or more pharmaceutical materials. In some embodiments, software 130 obtains data from database 120 and/or user(s) 150 (e.g., by uploading data). The database 120 may be of any suitable type (e.g., database system, multi-file, flat file, etc.) and may store data in any suitable way and in any suitable format, as aspects of the technology described herein are not limited in this respect. The database 120 may be part of software 130 (not shown) or excluded from software 130, as shown in FIG. 1. The database 120 may be part of or external to computing system 110.

In some embodiments, the stored data may have been previously uploaded by a user (e.g., user 150), and/or from one or more public data stores and/or studies. In some embodiments, a portion of the data may be processed by the data processing module 134 to obtain processed data. In some embodiments, a portion of the data may be processed by the feature generation module 136 to generate sets of features to be provided as input to a machine learning model. In some embodiments, a portion of the data may be used to train one or more machine learning models (e.g., with the model training module 138).

User interface module 144 may be a graphical user interface (GUI), a text-based user interface, and/or any other suitable type of interface through which a user may provide input and view information generated by software 130. For example, in some embodiments, the user interface may be a webpage or web application accessible through an Internet browser. In some embodiments, the user interface may be a graphical user interface (GUI) of an app executing on the user's mobile device. In some embodiments, the user interface may include a number of selectable elements through which a user may interact. For example, the user interface may include dropdown lists, checkboxes, text fields, or any other suitable element.

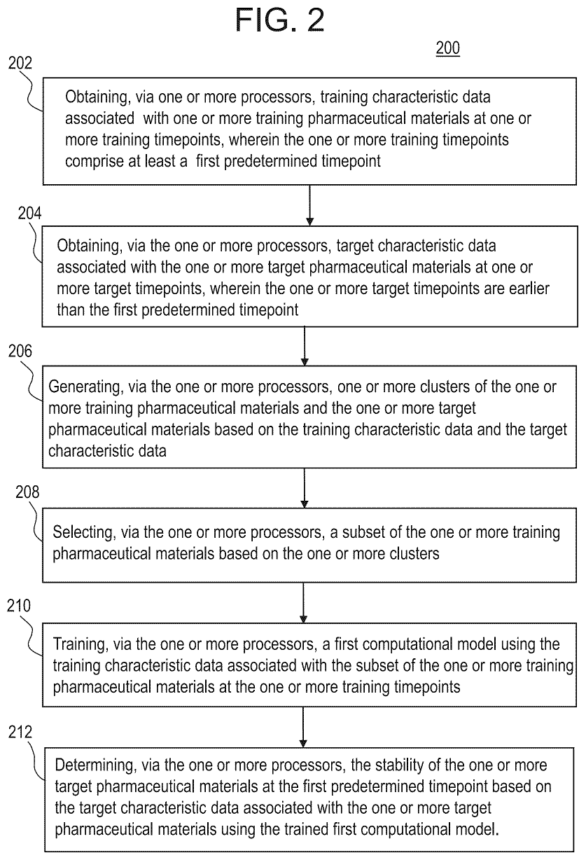

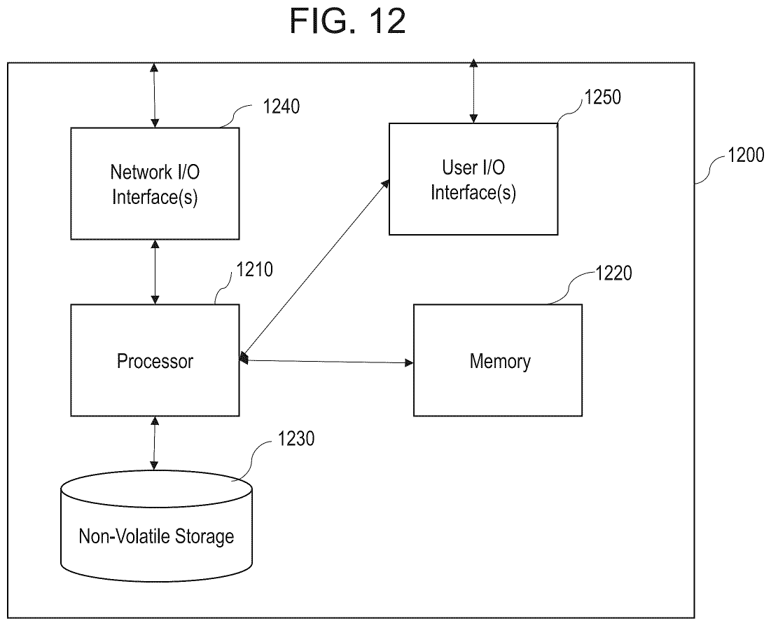

FIG. 2A is a flowchart of an illustrative method 200 for determining stability of one or more target pharmaceutical materials, in accordance with some embodiments of the technology described herein. One or more steps of method 200 may be performed automatically by any suitable computing system(s). For example, the step(s) may be performed by a laptop computer, a desktop computer, one or more servers, in a cloud computing environment, computer system 100, and computing device 1200 as described herein within respect to FIG. 12, and/or in any other suitable way. For example, in some embodiments, step 202 may be performed automatically by any suitable computing system(s) and/or device(s). As another example, step 204 may be performed automatically by any suitable computing system(s) and/or device(s).

Step 202 may include obtaining, via one or more processors, training characteristic data associated with one or more training pharmaceutical materials at one or more training timepoints. The one or more training timepoints can comprise at least a first predetermined timepoint. One example of step 202 can comprise obtaining high molecular weight (HMW) value of a small molecule pharmaceutical material under storage condition at 12 months from the beginning of storage. One or more training pharmaceutical materials can comprise a therapeutic product comprising an active pharmaceutical ingredient (API). A training pharmaceutical material may further comprise additional substances such as carriers or excipients. In some embodiments, a training pharmaceutical material is subject to regulation and premarket approval by a government regulatory agency, such as the Food and Drug Administration (FDA) or the European Medicines Agency (EMA). In some embodiments, a training pharmaceutical material is authorized for administration to a human subject by such a government regulatory agency. Examples of training pharmaceutical materials can include biological therapies, small synthetic molecules, and nucleic acids such as small interfering RNA (siRNA) and DNA. In some embodiments, the training pharmaceutical material is for medical use. In some embodiments, the training pharmaceutical material is for medical use in a human subject. The biological therapy can comprise a therapeutic composition comprising a biological macromolecule, for example a gene therapy, a therapeutic protein, a nucleic acid, a virus, or a cell or a portion thereof. The biological therapy can comprise an antibody, an antigen-binding antibody fragment, an antibody protein product, a Bi-specific T cell engager (BiTE®) molecule, a bispecific antibody, a trispecific antibody, an Fc fusion protein, a recombinant protein, a recombinant virus, a recombinant T cell, a synthetic peptide, deoxyribonucleic acid (DNA), ribonucleic acid (RNA), and an active fragment of a recombinant protein.

The number of one or more training timepoints can be at least 1, 2, 3, 4, 5, 6, 7, 8, 9, 10, 20, 30, 40, 50, 60, 70, 80, 90, or more timepoints. In some embodiments, the number of one or more training timepoints can be at most 90, 80, 70, 60, 50, 40, 30, 20, 10, or less timepoints. In some embodiments, the one or more training timepoints span a period of months or years. In some embodiments, one or more training timepoints span a period of months or years from the beginning of storage of one training pharmaceutical material. In some embodiments, the one or more training timepoints span at least 1 month, 2 months, 3 months, 4 months, 6 months, 7 months, 8 months, 9 months, 10 months, 11 months, 12 months, 18 months, 24 months, 30 months, 36 months or longer. In some embodiments, the one or more training timepoints span at most 36 months, 30 months, 24 months, 18 months, 12 months, 11 months, 10 months, 9 months, 8 months, 7 months, 6 months, 5 months, 4 months, 3 months or shorter. The one or more training timepoints can comprise at least a first predetermined timepoint. The first predetermined timepoint can comprise any timepoint within the one or more training timepoints. The first predetermined timepoint can comprise any timepoint. In some embodiments, the first predetermined timepoint can be at least 1 month, 2 months, 3 months, 4 months, 6 months, 7 months, 8 months, 9 months, 10 months, 11 months, 12 months, 18 months, 24 months, 30 months, 36 months, or longer. In some embodiments, the first predetermined timepoint can be at most 36 months, 30 months, 24 months, 18 months, 12 months, 11 months, 10 months, 9 months, 8 months, 7 months, 6 months, 5 months, 4 months, 3 months or shorter.

Any suitable analytical technique can be used for collecting training characteristic data with the methods described herein. Techniques for collecting training characteristic data can include mass spectrometry, chromatography, electrophoresis, spectroscopy, light obscuration, particle methods (nanoparticle/visible/micron-sized resonant mass or Brownian motion), analytical centrifugation, imaging and imaging characterizations, and immunoassays. Example techniques for collecting training characteristic data can include reduced and non-reduced peptide mapping (which may detect chemical modifications), chromatography (such as size exclusion chromatography (SEC), ion exchange chromatography (IEX) such as cation exchange chromatography (CEX), hydrophobic interaction chromatography (HIC), affinity chromatography such as Protein A-column chromatography, or reverse phase (RP) chromatography), capillary isoelectric focusing (cIEF), capillary zone electrophoresis (CZE), free flow fractionation (FFF), or ultracentrifugation (UC), HIAC (such as for detecting subvisible particle count), MFI (such as for detecting subvisible particle count and morphology), visible inspection (visible particles), SDS-PAGE (such as for detecting fragments, covalent aggregates), color analysis (Trp Ox), rCE-SDS and nrCE-SDS (such as for detecting fragments that are partial molecules), nanoparticle sizing methods, spectroscopy methods (such as FTIR, CD, intrinsic fluorescence, or ANS dye binding), an Ellman's assay (free sulfhydryl's), SEC-MALS, HILIC (glycan map), and ELISA (such as for detecting HCP).

Step 204 may include obtaining, via one or more processors, target characteristic data associated with one or more target pharmaceutical materials at one or more target timepoints. The one or more target timepoints can be earlier than the first predetermined timepoint. One example of step 204 can comprise obtaining HMW value of a small molecule drug under storage condition at 6 months from the beginning of storage. One or more target pharmaceutical material can comprise a therapeutic product comprising an active pharmaceutical ingredient (API). A target pharmaceutical material may further comprise additional substances such as carriers or excipients. In some embodiments, a target pharmaceutical material is subject to regulation and premarket approval by a government regulatory agency, such as the Food and Drug Administration (FDA) or the European Medicines Agency (EMA). In some embodiments, a target pharmaceutical material is authorized for administration to a human subject by such a government regulatory agency. Examples of target pharmaceutical materials can include biological therapies, small synthetic molecules, and nucleic acids such as small interfering RNA (siRNA) and DNA. In some embodiments, the target pharmaceutical material is for medical use. In some embodiments, the target pharmaceutical material is for medical use in a human subject. The biological therapy can comprise a therapeutic composition comprising a biological macromolecule, for example a gene therapy, a therapeutic protein, a nucleic acid, a virus, or a cell or a portion thereof. In some embodiments, the one or more training pharmaceutical materials are different from the one or more target pharmaceutical materials. In some embodiments, the one or more training pharmaceutical materials are the same as the one or more target pharmaceutical materials. The biological therapy can comprise an antibody, an antigen-binding antibody fragment, an antibody protein product, a Bi-specific T cell engager (BiTE®) molecule, a bispecific antibody, a trispecific antibody, an Fc fusion protein, a recombinant protein, a recombinant virus, a recombinant T cell, a synthetic peptide, deoxyribonucleic acid (DNA), ribonucleic acid (RNA), and an active fragment of a recombinant protein.

The number of the one or more target timepoints can be at least 1, 2, 3, 4, 5, 6, 7, 8, 9, 10, 20, 30, 40, 50, 60, 70, 80, 90, or more timepoints. In some embodiments, the number of the one or more target timepoints can be at most 90, 80, 70, 60, 50, 40, 30, 20, 10, or less timepoints. In some embodiments, the one or more target timepoints span a period of months or years. In some embodiments, the one or more target timepoints span a period of months or years from the beginning of storage of one target pharmaceutical material. In some embodiments, the one or more target timepoints span at least 1 month, 2 months, 3 months, 4 months, 6 months, 7 months, 8 months, 9 months, 10 months, 11 months, 12 months, 18 months, 24 months, 30 months, 36 months or longer. In some embodiments, the one or more target timepoints span at most 36 months, 30 months, 24 months, 18 months, 12 months, 11 months, 10 months, 9 months, 8 months, 7 months, 6 months, 5 months, 4 months, 3 months or shorter. The one or more target timepoints can be earlier than the first predetermined timepoint. For instance, if the first predetermined timepoint is 24 months, and the one or more target timepoints can be any time earlier than 24 months (e.g., 3 months, 6 months, 12 months).

Any suitable analytical technique can be used for collecting target characteristic data with the methods described herein. Techniques for collecting target characteristic data can include mass spectrometry, chromatography, electrophoresis, spectroscopy, light obscuration, particle methods (nanoparticle/visible/micron-sized resonant mass or Brownian motion), analytical centrifugation, imaging and imaging characterizations, and immunoassays. Example techniques for collecting target characteristic data can include reduced and non-reduced peptide mapping (which may detect chemical modifications), chromatography (such as size exclusion chromatography (SEC), ion exchange chromatography (IEX) such as cation exchange chromatography (CEX), hydrophobic interaction chromatography (HIC), affinity chromatography such as Protein A-column chromatography, or reverse phase (RP) chromatography), capillary isoelectric focusing (cIEF), capillary zone electrophoresis (CZE), free flow fractionation (FFF), or ultracentrifugation (UC), HIAC (such as for detecting subvisible particle count), MFI (such as for detecting subvisible particle count and morphology), visible inspection (visible particles), SDS-PAGE (such as for detecting fragments, covalent aggregates), color analysis (Trp Ox), rCE-SDS and nrCE-SDS (such as for detecting fragments that are partial molecules), nanoparticle sizing methods, spectroscopy methods (such as FTIR, CD, intrinsic fluorescence, or ANS dye binding), an Ellman's assay (free sulfhydryl's), SEC-MALS, HILIC (glycan map), and ELISA (such as for detecting HCP)

The training characteristic data can comprise one or more training characteristic categories. The one or more training characteristic categories can comprise at least one of a high molecular weight value (e.g., a high molecular weight percentage, which can represent formation of high molecular weight (HMW) species during drug product storage under recommended storage conditions), a concentration, or a pH (or pH value). In some embodiments, the one or more training characteristic categories can comprise at least one of pH value, high molecular weight, subvisible particle number, low molecular weight, medium molecular weight, average molecular weight, fill volume, isoelectric point, extinction coefficient, net charge, cysteine count, chain length, deamidation status, deamination status, cyclization status, oxidation status, isomerization status, fragmentation/clipping, N-terminal and C-terminal variants, reduced and partial species, folded structure, monoclonal antibodies (mAb) characteristics (e.g., charge variants), number of disulfide bonds, surface charge distribution, glycosylation, hydrophobic interactions, hydrogen bond interactions, Pi-Pi stacking, ionic and charge interactions, van der Waals interactions, post translational modifications, or surface hydrophobicity.

When training characteristic data comprises one or more training characteristic categories, the method can further comprise, prior to obtaining target characteristic data associated with the one or more target pharmaceutical materials at one or more target timepoints, selecting a subset of the one or more training characteristic categories based on one or more predetermined criteria. For example, the one or more training characteristic categories can comprise a high molecular weight value, a concentration, and a pH value, and the subset of the one or more training characteristic categories can comprise a high molecular weight value and a concentration. The one or more predetermined criteria can be set by a user based on the user's professional judgment. The one or more predetermined criteria can comprise storage temperature (e.g., choosing training characteristic data at a specific temperature like 5° C.), an analytical technique (e.g., choosing training characteristic data via SE-HPLC), and form of training pharmaceutical materials (e.g., using pharmaceutical materials in liquid form instead of solid form). The selected subset of the one or more training characteristic categories can be the features that generated to input to the first computational model or the second computational model. For instance, the subset of the one or more training characteristic categories can comprise a high molecular weight value and a concentration, then the high molecular weight value and the concentration can be two features that will be input to the first computational model.

The target characteristic data can comprise one or more target characteristic categories. The one or more target characteristic categories can comprise at least one of a high molecular weight value, a concentration, or a pH. In some embodiments, the one or more target characteristic categories can comprise at least one of pH value, high molecular weight, subvisible particle number, low molecular weight, medium molecular weight, average molecular weight, fill volume, isoelectric point, extinction coefficient, net charge, cysteine count, chain length, deamidation status, deamination status, cyclization status, oxidation status, isomerization status, fragmentation/clipping, N-terminal and C-terminal variants, reduced and partial species, folded structure, monoclonal antibodies (mAb) characteristics (e.g., charge variants), number of disulfide bonds, surface charge distribution, glycosylation, hydrophobic interactions, hydrogen bond interactions, Pi-Pi stacking, ionic and charge interactions, van der Waals interactions, post translational modifications, or surface hydrophobicity.

When target characteristic data comprise one or more target characteristic categories, the method can further comprise, prior to obtaining target characteristic data associated with the one or more target pharmaceutical materials at one or more target timepoints, selecting a subset of the one or more target characteristic categories based on one or more predetermined criteria. For example, the one or more target characteristic categories can comprise a high molecular weight value, a concentration, and a pH value, and the subset of the one or more target characteristic categories can comprise a high molecular weight value and a concentration. The one or more predetermined criteria can be set by a user based on the user's professional judgment. In some embodiments, the one or more target characteristic categories are the same as the one or more training characteristic categories. In some embodiments, the one or more target characteristic categories are different from the one or more training characteristic categories. The selected subset of the one or more target characteristic categories can be the features that generated to input to the first computational model or the second computational model. For instance, the subset of the one or more target characteristic categories can comprise a high molecular weight value and a concentration, then the high molecular weight value and the concentration can be two features that will be input to the first computational model.

Step 206 can comprise generating, via the one or more processors, one or more clusters of the one or more training pharmaceutical materials and the one or more target pharmaceutical materials based on the training characteristic data and the target characteristic data. The number of one or more clusters can be at least 1, 2, 3, 4, 5, 10, 15, 20, 50, 100 or larger. The number of one or more clusters can be at most 100, 50, 20, 10, 5, or smaller.

In some embodiments, one cluster of the one or more training pharmaceutical materials and the one or more target pharmaceutical materials comprise a subset of one or more training pharmaceutical materials and one or more target pharmaceutical materials. For instance, the number of one or more training pharmaceutical materials is 7 and the number of one or more target pharmaceutical materials is 1, and one cluster can comprise 6 training pharmaceutical materials and 1 target pharmaceutical material. In another example, the number of one or more training pharmaceutical materials is 7 and the number of one or more target pharmaceutical materials is 1, and one cluster can comprise 5 training pharmaceutical materials and 1 target pharmaceutical material. In some embodiments, one or more training pharmaceutical materials in a cluster have same or similar characteristic data as one target pharmaceutical material in the cluster. For example, the pH values of one or more training pharmaceutical materials in a cluster can fall within [−1, +1] of the pH value of one target pharmaceutical material in the same cluster. In this example, in one cluster, the pH value of the target pharmaceutical material is 5, and the pH values of one or more training pharmaceutical materials in a cluster can fall within [4, 6]. In another example, the percentage of high molecular weight of one or more training pharmaceutical materials in a cluster can be similar to the percentage of high molecular weight of one target pharmaceutical material in the same cluster.

The generating the one or more clusters of the one or more training pharmaceutical materials and the one or more target pharmaceutical materials can comprise using at least one of a dimensionality reduction technique or a clustering technique. The dimensionality reduction technique can comprise principal component analysis (PCA), factor analysis, linear discriminant analysis, singular value decomposition, kernel principal component analysis, T-distributed stochastic neighbor embedding, multi-dimensional scaling, or isometric mapping. The clustering technique can comprise centroid-based clustering, density-based clustering, distribution-based clustering, or hierarchical clustering. For instance, principal component analysis can be performed prior to applying an unsupervised mean shift clustering method.

The generating the one or more clusters of the one or more training pharmaceutical materials and the one or more target pharmaceutical materials can comprise using at least one of a dimensionality reduction technique or a clustering technique to group a set of data points into clusters, and the data points within the same cluster share similar characteristics while data points in different clusters are dissimilar. The clustering technique can comprise different clustering algorithms, including as K-Means, Hierarchical Clustering, or DBSCAN. K-Means clustering can specify the number of clusters (k) in advance. The clustering algorithm can then assign data points to k clusters by minimizing the distance between data points and the cluster centroids, and such process can iterate until the clusters stabilize. The clustering algorithms can compute distances between data points and iteratively assign points to clusters based on a similarity metric (such as Euclidean distance for K-Means).

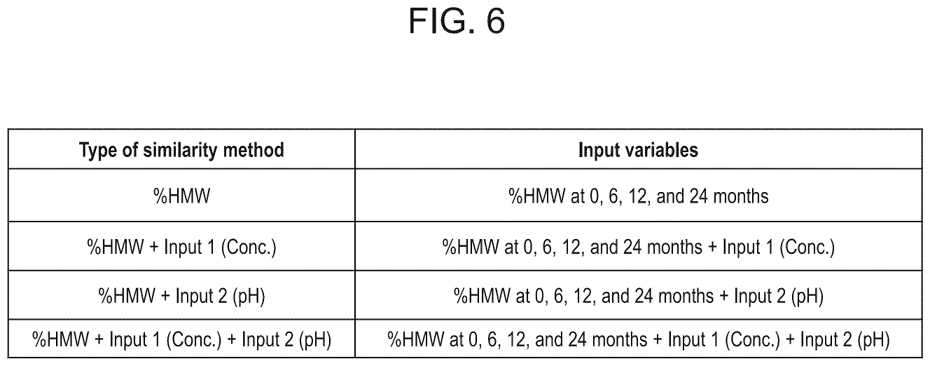

FIG. 6 depicts exemplary methods used for the assessment of molecule similarity. As shown in FIG. 6, different combinations of one or more characteristic categories are used. For instance, in the first row, the HMW values up to 24 months can be used. In other examples, the HMW values and pH, the HMW values and concentration, or the HMW values and pH and concentration can be used. The different combination of one or more characteristic categories can be used as inputs to the dimensionality reduction and the clustering technique to generate one or more clusters, which may indicate similar molecules. In one example, the case of the first row of FIG. 6, HMW values at 0, 6, 12, 24 months are used as inputs to the dimensionality reduction and the clustering technique to generate one or more clusters. In another example, the last row of FIG. 6, HMW values along with pH and concentration can be used inputs to the dimensionality reduction and the clustering technique to generate one or more clusters.

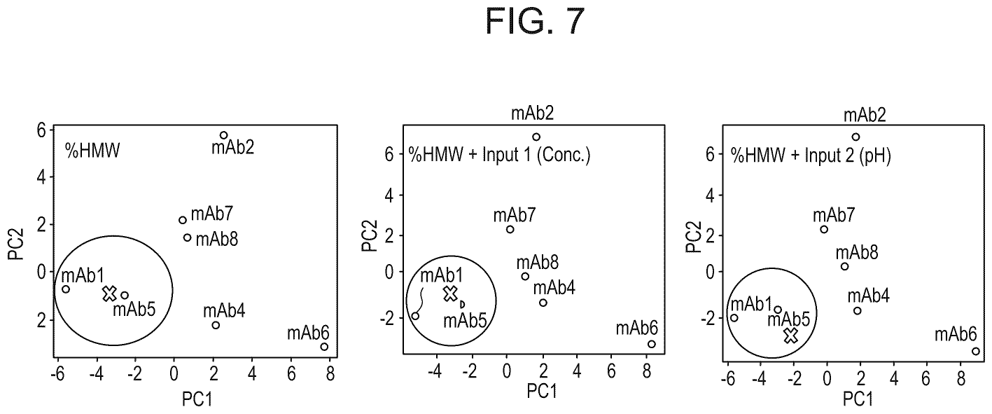

FIG. 7 shows exemplary results from one clustering technique using data from different combinations of training characteristic categories and target characteristic categories. The plots here show the results from meanshift clustering using different combinations of training characteristic categories and target characteristic categories. For instance, the plot on the left shows the results from meanshift clustering using the HMW value associated with the training pharmaceutical materials and the targeting pharmaceutical material; the plot in the middle shows the results from meanshift clustering using the combination of the HMW value and concentration associated with the training pharmaceutical materials and the targeting pharmaceutical material; and the plot on the right shows the results from meanshift clustering using the combination of the HMW value and pH value associated with the training pharmaceutical materials and the targeting pharmaceutical material. In the x and y axis, principal component 1 (PC1) and principal component 2 (PC2) are identified from PCA. PC1 and PC2 can be some linear combination of characteristic categories that are used as inputs to or obtained from a dimensionality reduction technique or a clustering technique, or any type of mathematical methods. These plots in FIG. 7 show how the training pharmaceutical materials are arranged with respect to the target pharmaceutical material denoted by the cross symbol. The closer the points associated with the training pharmaceutical materials to the cross symbol, the more similar the training pharmaceutical materials are to the target pharmaceutical material. In the plots of FIG. 7, mAb1 (monoclonal antibody 1) and mAb5 are closer to the target pharmaceutical material whereas mAb2 and mAb6 are the farthest, and mAb4, mAb7 and mAb8 lie somewhere in between. This shows that mAb1 and mAb5 are more similar to the target pharmaceutical material followed by mAb4, mAb7 and mAb8.

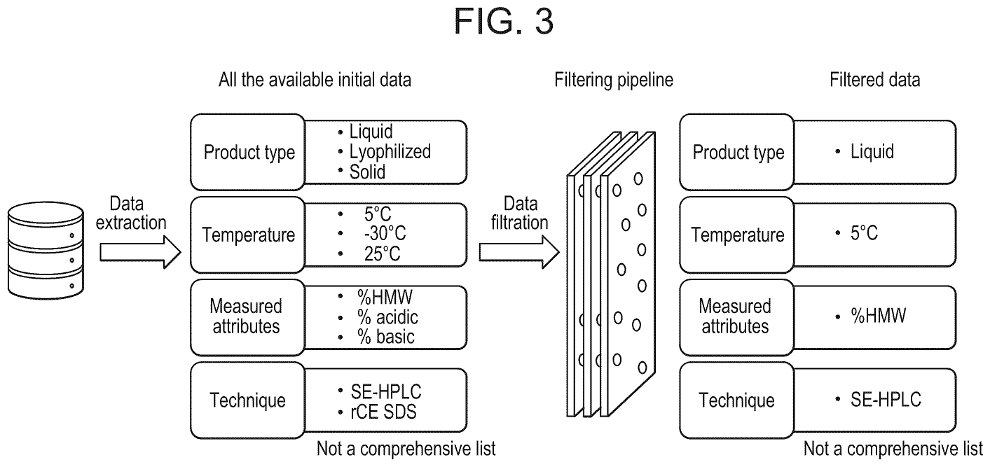

Prior to step 206, the method can further comprise a step of data selection (e.g., selecting training characteristic data associated with one or more training pharmaceutical materials or target characteristic data associated with the one or more target pharmaceutical materials). As shown in FIG. 3, the data selection can comprise at least one of data extraction, data filtering, data cleaning, or data pre-processing. In case of predicting stability (e.g., the HMW values), the input data (e.g., characteristic data associated with one or more pharmaceutical materials) can comprise metrics or variables that can include, affect and/or influence the stability indicating attribute (e.g., the HMW values). One or more characteristic categories can be selected to filter the data. Such one or more characteristic categories can comprise anything that can impact stability of the pharmaceutical material, including, but not limited to, modality, prior data (e.g., prior data associated with stability indicating attributes) corresponding to pharmaceutical materials (e.g., monoclonal antibodies (mAbs)), storage conditions/status, container closure, container orientation, test methods, stability data to shelf-life, temperature, pH, concentration, and formulation. In FIG. 3, once the data is extracted from database 120 or data extraction module 132, one or more data cleaning and data filtering steps can be performed to make the data consistent and uniform. In one example, the data flows from a data lake and then is filtered using one or more selected characteristic categories, such as product type, temperature, measured attributes, or techniques. Data cleaning can also be performed to make the data clear, contextual, and integrated.

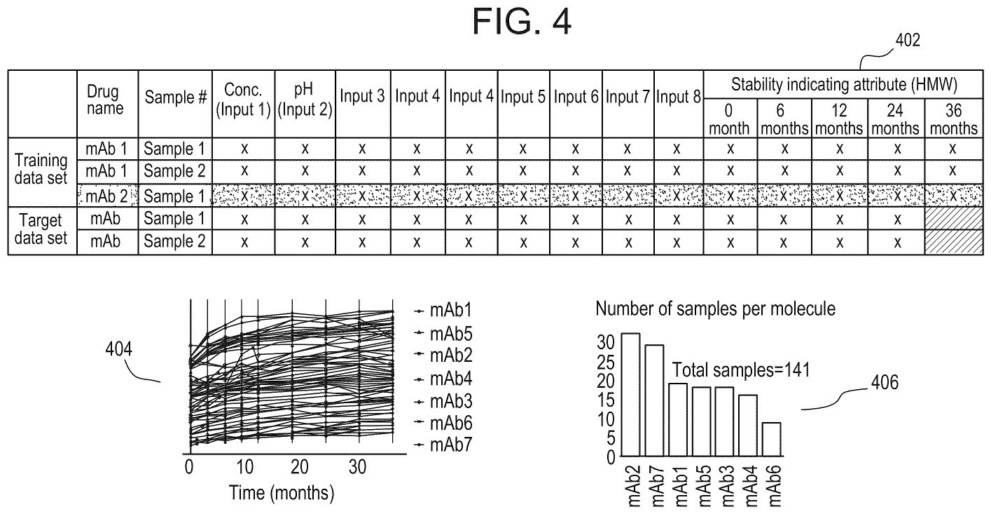

FIG. 4 depicts an exemplary table 402 showing characteristic data of one or more pharmaceutical materials, an exemplary plot 404 showing stability indicating attribute (e.g., high molecular weight (HMW)) profiles of one or more pharmaceutical materials, and an exemplary plot 406 showing the number of lots (e.g., samples) of one or more pharmaceutical materials. The characteristic data of pharmaceutical materials can comprise one or more characteristic categories including concentration, pH, HMW values measured at different timepoints (e.g., 6 months, 12 months, 24 months, and 36 months). The training characteristic data and/or stability of the training pharmaceutical materials can comprise the 36-month HMW value, which is used to predict target characteristic data of target pharmaceutical materials at 36 months. The model (e.g., machine learning model) can learn the relationships between the 36-month HMW values and all these inputs and use those learning to project the 36-month values of the stability indicating attributes (e.g., HMW values) for target pharmaceutical materials. Plot 404 shows the stability indicating attribute profile for training characteristic data of the training pharmaceutical materials, wherein the y-axis demonstrates the value of the stability indicating attribute and x-axis demonstrates the time. Plot 404 shows that majority of profiles follow a non-linear trend. Plot 406 shows 7 different training pharmaceutical materials and each of them has multiples lots (samples) as shown by the distribution here.

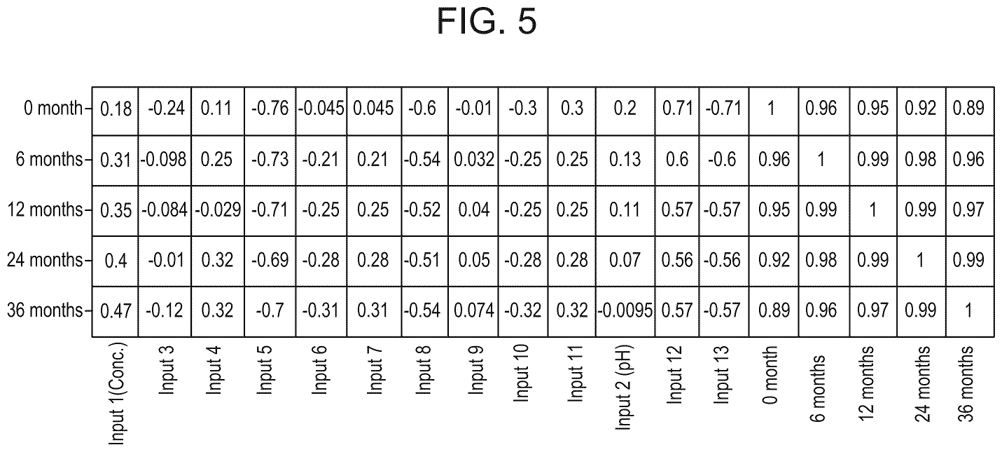

Following data selection step (e.g., data extraction and filtration), the method can further comprise performing data analysis to identify any relationships between the input characteristic data of pharmaceutical material and the HMW values. As shown in FIG. 5, the table shows the correlation between the input characteristic data of pharmaceutical material (e.g., training characteristic data of training pharmaceutical material) and the HMW values. The columns of the table represent different training characteristic categories, and the row of the table represent different HMW values measured at different timepoints. The values in the table range between −1 and 1 with 1 representing complete positive correlation and −1 representing negative correlation. As shown in FIG. 5, all the HMW values are positively correlated with each other. Besides, training characteristic data of the training pharmaceutical materials are both positively and negatively correlated with the HMW values. For instance, concentration is positively correlated and pH—it affects the HMW values positively but it is weakly correlated with it as seen from the values, which are closer to zero.

Step 208 can comprise selecting, via the one or more processors, a subset of the one or more training pharmaceutical materials based on the one or more clusters. As described elsewhere herein, in some embodiments, one or more training pharmaceutical materials in a cluster have same or similar characteristic data as one target pharmaceutical material in the cluster. In this case, selecting the subset of the one or more training pharmaceutical materials comprises selecting the one or more training pharmaceutical materials in the cluster. For instance, if the number of the one or more training pharmaceutical materials is 8, and the number of the one or more training pharmaceutical materials in the cluster is 6, then selecting the subset of the one or more training pharmaceutical materials comprises selecting the 6 pharmaceutical materials in the cluster. FIG. 6, as described elsewhere herein, depicts exemplary methods used for the assessment of molecule similarity.

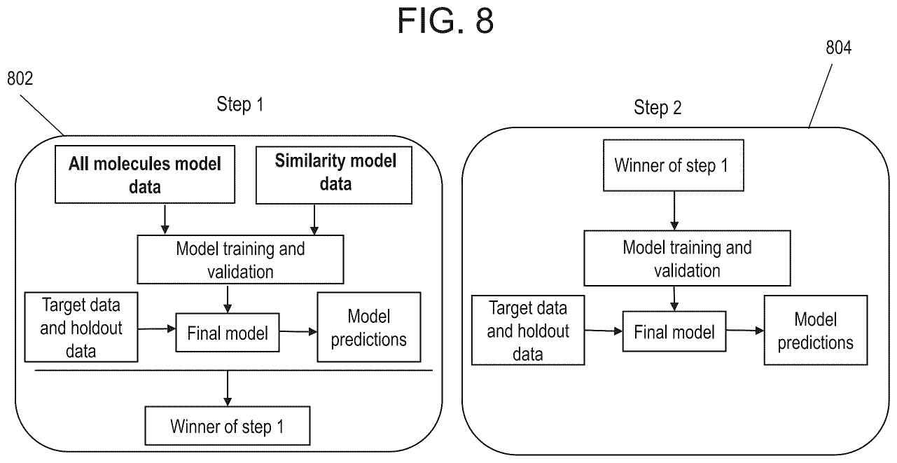

Step 210 can comprise training, via the one or more processors, a first computational model using the training characteristic data associated with the subset of the one or more training pharmaceutical materials at the one or more training timepoints. The first computational model trained with the training characteristic data associated with the subset of the one or more training pharmaceutical materials at the one or more training timepoints can be a similarity model. The similarity model can be trained with similarity model data, as illustrated in FIG. 8. The first computational model can comprise any type of mathematical, statistical, or machine learning model, including, but not limited to, a supervised machine learning model, an unsupervised machine learning model, a reinforcement learning model, a regression model (e.g., a logistic regression model, a multinomial logistic regression model), a support vector machine model, a multilayer perceptron model, a random forest model, a natural language processing model, a neural network model, a cluster model, and a dimensionality reduction model. The training can comprise inputting the training characteristic data associated with the subset of the one or more training pharmaceutical materials at the one or more training timepoints into the first computational model and adjusting parameters and hyperparameters.

When training the first computational model, a validation process can be performed. The validation process can comprise any type of validation technique, including, but not limited to, cross-validation, k-fold cross validation, leave-one-out cross-validation, bootstrapping, Monte Carlo cross-validation, holdout validation, and shuffle split. The validation process can be used to tune the hyperparameters. For example, a predefined range of value for each of the hyperparameters can be assumed, and then for each possible combination of the hyperparameter values, models can be built iteratively in such a way one of one or more training pharmaceutical materials is left out for validation purposes (e.g., leave-one-out cross validation). In some cases, if training characteristic data of 7 training pharmaceutical materials are used to train a first computational model, in each iteration, a computational model can be built using training characteristic data of 6 out of 7 training pharmaceutical materials and with the remaining training characteristic data of 1 training pharmaceutical material as validation. Once the model is built, predictions can be made for the pharmaceutical material that is left out and compared with its actual value. This procedure can be repeated for all possible combinations of hyperparameter values in the predefined range. The combination with the lowest error can be chosen for the given dataset. Once the hyperparameters are determined, the functional form of the first computational model can be set, and the model parameters can be obtained using an iterative optimization procedure. Since the results of an optimization procedure depend on the initial values, the procedure can be repeated several times to account for these differences. In some embodiments, the differences can be used to calculate a confidence interval (e.g., 95% confidence interval) of the predictions of the first computational model.

Step 212 can comprise determining, via the one or more processors, the stability of the one or more target pharmaceutical materials at the first predetermined timepoint based on the target characteristic data associated with the one or more target pharmaceutical materials using the trained first computational model. The stability of the one or more target pharmaceutical materials can comprise values of one or more stability indicating attributes, for example, concentrations, pH values, or high molecular weight values (e.g., a percentage of high molecular weight) of the one or more target pharmaceutical materials at the first predetermined timepoint.

The one or more training timepoints can further comprise a second predetermined timepoint. The second predetermined timepoint can comprise any timepoint within the one or more training timepoints. For instance, the second predetermined timepoint can comprise 1 month, 2 months, 3 months, 4 months, 6 months, 7 months, 8 months, 9 months, 10 months, 11 months, 12 months, 18 months, 24 months, 30 months, or 36 months. The first predetermined timepoint can be earlier than the second predetermined timepoint. For instance, if the first predetermined timepoint is 24 months, then the second predetermined timepoint can be any timepoint later than 24 months, such as 30 months or 36 months. The method can further comprise determining the stability of the one or more target pharmaceutical materials based on the target characteristic data associated with the one or more target pharmaceutical materials at the second predetermined timepoint using the trained first computational model or any type of model. For instance, if the second predetermined timepoint is 36 months, then the method can further comprise determining the stability of the one or more target pharmaceutical materials based on the target characteristic data associated with the one or more target pharmaceutical materials at 36 months using the trained first computational model or any type of mathematical or computational model.

The method can further comprise causing a display (e.g., display 116) to present a visual indication of the determined stability. The method can include generating and/or populating a user interface, for example. The determined stability can be shown as a high molecular weight percentage over time. In this situation, the visual indication of the determined stability can be presented on a user interface (e.g., display 116) to users (e.g., research professionals), and users can review the determined stability and provide input (e.g., recommendations of how to adjust storage status) via user input device (e.g., user input device 118) based on the determined stability.

The method can further comprise generating one or more recommendations to adjust at least one storage status associated with the one or more target pharmaceutical materials based on the determined stability. In some embodiments, the one or more recommendations can be generated via any type of computational, mathematical, statistical, or machine learning model, including, but not limited to, a supervised machine learning model, an unsupervised machine learning model, a reinforcement learning model, a regression model (e.g., a logistic regression model, a multinomial logistic regression model), a support vector machine model, a multilayer perceptron model, a random forest model, a natural language processing model, a neural network model, a cluster model, and a dimensionality reduction model. After training the second computational model, a validation process, as described elsewhere herein, can be performed. In some embodiments, the one or more recommendations can be generated by a user who has reviewed the determined stability via a display.

The method can further comprise adjusting, via the one or more processors, at least one storage status associated with the one or more target pharmaceutical materials based on the determined stability and/or one or more recommendations. The storage status can comprise a humidity, a temperature, a buffer solution, a storage container, a container orientation, and an air quality related to how to store the one or more target pharmaceutical materials. Such adjusting can be achieved automatically or manually. In some embodiments, if the determined stability of one target pharmaceutical material with certain storage status is lower than a threshold (e.g., 30% high molecular weight), then such storage status can be adjusted automatically via predetermined algorithm to see whether the high molecular weight can decrease. For instance, if the determined stability of one target pharmaceutical material at room temperature at the first predetermined timepoint is lower than a threshold (e.g., 30% high molecular weight), then the temperature can be adjusted automatically via predetermined algorithm to a lower temperature than the room temperature to see whether the high molecular weight can decrease. Such predetermined algorithm can be used to generate one or more recommendations to adjust at least one storage status. In some embodiments, if the determined stability of one target pharmaceutical material with certain storage status at the first predetermined timepoint is lower than a threshold (e.g., 30% high molecular weight), then the determined stability can be used to generate a recommendation of adjusting such storage status. Such recommendation can be sent to a user interface or user input device (e.g., user input device 118), so a user can review the recommendation, and approve or edit the recommendation for adjusting the storage status. Then, the storage status can be adjusted based on the approved or edited recommendation. For instance, if the determined stability of one target pharmaceutical material at room temperature at the first predetermined timepoint is lower than a threshold (e.g., 30% high molecular weight), then the determined stability can be used to generate a recommendation of adjusting the temperature. Such recommendation can be sent to a user interface or user input device (e.g., user input device 118), so a user can review the recommendation, and approve or edit the recommendation for adjusting the temperature to see whether the high molecular weight can decrease.

The method can further comprise training a second computational model using the training characteristic data associated with the one or more training pharmaceutical materials at the one or more training timepoints (e.g., all molecules model). The second computational model trained using training characteristic data associated with the one or more training pharmaceutical materials at the one or more training timepoints can be an all molecules model. The all molecules model can be trained with all molecules model data, as illustrated in FIG. 8. In some embodiments, the second computational model is the same as the first computational model. In some embodiments, the second computational model is different from the first computational model. The second computational model can comprise any type of mathematical, statistical, or machine learning model, including, but not limited to, a supervised machine learning model, an unsupervised machine learning model, a reinforcement learning model, a regression model (e.g., a logistic regression model, a multinomial logistic regression model), a support vector machine model, a multilayer perceptron model, a random forest model, a natural language processing model, a neural network model, a cluster model, and a dimensionality reduction model. After training the second computational model, a validation process, as described elsewhere herein, can be performed.

In some embodiments, a neural network model/classifier may be used. The neural network classifier may be trained using any suitable neural network optimization software. The optimization software may be configured to perform neural network training by gradient descent, stochastic gradient descent, or in any other suitable way. In some embodiments, a cluster model may be used. The cluster model can find natural groupings in a dataset. To identify natural groupings, measure similarity (or dissimilarity) between two samples maybe determined based on a distance function and to compute the matrix of distances between all pairs of samples in the training set. Once a method for measuring “similarity” or “dissimilarity” between points in a dataset is selected, clustering can use a criterion function that measures the clustering quality of any partition of the data. Partitions of the data set that extremize the criterion function are used to cluster the data. In some embodiments, principal component analysis (PCA) algorithms, one type of dimensionality model may be used. Principal components (PCs) can be uncorrelated and can be ordered such that the kth PC has the kth largest variance among PCs. The kth PC can be interpreted as the direction that maximizes the variation of the projections of the data points such that it is orthogonal to the first k-1 PCs. The first few PCs can capture most of the variation in a training set. In contrast, the last few PCs can often be assumed to capture only the residual ‘noise’ in the training set. In some embodiments, a support vector machine (SVM) model can be used. When used for classification, SVMs can separate a given set of binary labeled data training set with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space can correspond to a non-linear decision boundary in the input space.

The method can further comprise comparing, via the one or more processors, the trained first computational model and the trained second computational model based on a level of prediction accuracy. The level of prediction accuracy can comprise an error metric, which indicates a prediction error. The lower the value, the better the performance of the model. In some embodiments, the level of prediction accuracy can be negatively correlated with the error metric or the prediction error, which means the lower the prediction error, the higher the level of prediction accuracy. The error metric or prediction error of the trained first computational model can be generated based on the difference or comparison between an output of the trained first computational model (e.g., value of the stability indicating attribute) and an actual value of the stability indicating attribute obtained via wetlab/experimental processes. The error metric or prediction error of the trained second computational model can be generated based on the difference or comparison between an output of the trained second computational model (e.g., value of the stability indicating attribute) and an actual value of the stability indicating attribute obtained via wetlab/experimental processes. In this situation, by comparing the trained first computational model and the trained second computational model based on the prediction error, either trained first computational model or the trained second computational model can be chosen for determining the stability of the one or more target pharmaceutical materials. For example, if the prediction error of the trained first computational model is higher than the prediction error of the trained second computational model, then the second computational model can be chosen for determining the stability of the one or more target pharmaceutical materials. In another example, if the prediction error of the trained first computational model is lower than the prediction error of the trained second computational model, then the first computational model can be chosen for determining the stability of the one or more target pharmaceutical materials.

The method can further comprise determining, via the one or more processors, the stability of the one or more target pharmaceutical materials based on the comparison between the trained first computational model and the trained second computational model and the target characteristic data associated with the one or more target pharmaceutical materials at the first predetermined timepoint. The comparison between the trained first computational model and the trained second computational model can demonstrate which trained computational model works better. For example, if the prediction error of the trained first computational model is higher than the prediction error of the trained second computational model and then the second computational model is chosen for determining the stability of the one or more target pharmaceutical materials, the method can further comprise determining the stability of the one or more target pharmaceutical materials at the first predetermined timepoint based on the trained second computational model and the target characteristic data associated with the one or more target pharmaceutical materials. The method can further comprise determining, via the one or more processors, the stability of the one or more target pharmaceutical materials at the second predetermined timepoint based on the comparison between the trained first computational model and the trained second computational model and the target characteristic data associated with the one or more target pharmaceutical materials. For example, if the prediction error of the trained first computational model is higher than the prediction error of the trained second computational model and then the second computational model is chosen for determining the stability of the one or more target pharmaceutical materials, the method can further comprise determining the stability of the one or more target pharmaceutical materials at the second predetermined timepoint based on the trained second computational model and the target characteristic data associated with the one or more target pharmaceutical materials.

FIG. 8 is a diagram depicting an illustrative method for predicting stability of one or more target pharmaceutical materials. The method can comprise two steps. In step 1 802, two datasets are used to train different models. The first dataset can comprise the training characteristic data of the one or more training pharmaceutical materials (e.g., all molecules model data), and can be used to train the second computational model (e.g., an all molecules model). The second dataset can comprise the training characteristic data associated with the subset of the one or more training pharmaceutical materials (e.g., similarity model data) and can be used to train the first computational model (e.g., a similarity model). The subset of the one or more training pharmaceutical materials can be similar pharmaceutical materials to the target pharmaceutical materials based on the dimensionality reduction technique and clustering technique (e.g., as illustrated in FIG. 7).

As illustrated in FIG. 8, either the trained first computational model or the trained second computational model can be the final model. The trained first computational model and the trained second computational model can be used to predict the stability at the first predetermined timepoint (e.g., 24 months). In one example, experimental 24 months values for stability indicating attributes are available for the one or more target pharmaceutical materials, thus, for each model, a level of prediction accuracy can be determined between the experimental 24 months values for stability indicating attributes associated with the one or more target pharmaceutical materials and the predicted 24 months values for stability indicating attributes associated with the one or more target pharmaceutical materials. If, a level of prediction accuracy associated the trained first computational model is higher than a level of prediction accuracy associated the trained second computational model, then the trained first computational model (e.g., final model) can be chosen to predict stability at a second predetermined timepoint (e.g., 36-month) in step 2 804. In some other embodiments, the step 1 802 can also select which datasets (e.g., all molecules model data or similarity model data) to be used to train/predict the stability of the target pharmaceutical materials. For instance, if a model trained with the training characteristic data of the one or more training pharmaceutical materials (e.g., all molecules model data) show a higher level of prediction accuracy, then in step 2 804, the training characteristic data of the one or more training pharmaceutical materials can be used with the model to predict 36-month values for stability indicating attributes associated with the one or more target pharmaceutical materials. If a model trained the subset of training characteristic data of the one or more training pharmaceutical materials (e.g., similarity model data) show a higher level of prediction accuracy, then in step 2 804, the subset of training characteristic data of the one or more training pharmaceutical materials can be used with the model to predict 36-month values for stability indicating attributes associated with the one or more target pharmaceutical materials.

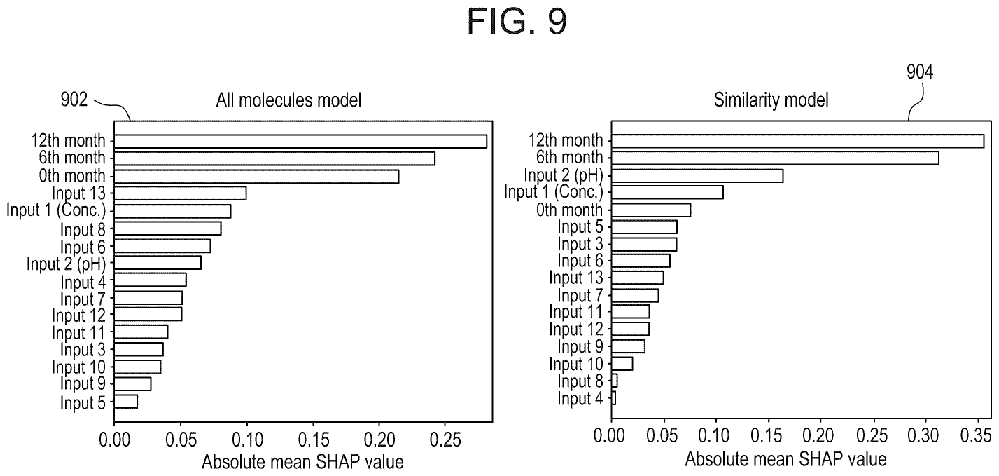

The methods and systems disclosed herein can comprise a SHAP analysis, comprising ranking the one or more training/target characteristic categories based on a level of impact on the determined stability. The higher rank can show the greater impact of the training/target characteristic categories on the determined stability. In some embodiments, the level of impact can comprise a numeric value, where the larger numeric value shows the greater impact of the training/target characteristic categories on the determined stability. FIG. 9 depicts exemplary diagrams depicting the impact of each training/target characteristic category (or input) on the stability predictions. In FIG. 9, x-axis shows SHAP value and y-axis shows the names of each training/target characteristic categories. Training/target characteristic categories on the top can have greater impact on the stability prediction than the training/target characteristic categories on the bottom. According to the rank, the impact of training/target characteristic categories can decrease from top to bottom. In the model trained with training characteristic data associated with one or more training pharmaceutical materials (e.g., all molecules model), shown as plot 902, the training characteristic categories with the highest impact comprise the HMW values at earlier timepoints. In the model trained with training characteristic data associated with a subset of one or more training pharmaceutical materials (e.g., similarity model), shown as plot 904, the training characteristic categories with the highest impact comprise the HMW values at earlier timepoints. Besides that, in both plots 902 and 904, concentration and pH impact the stability predictions, and pH has a larger impact in the plot 904 compared to the plot 902.

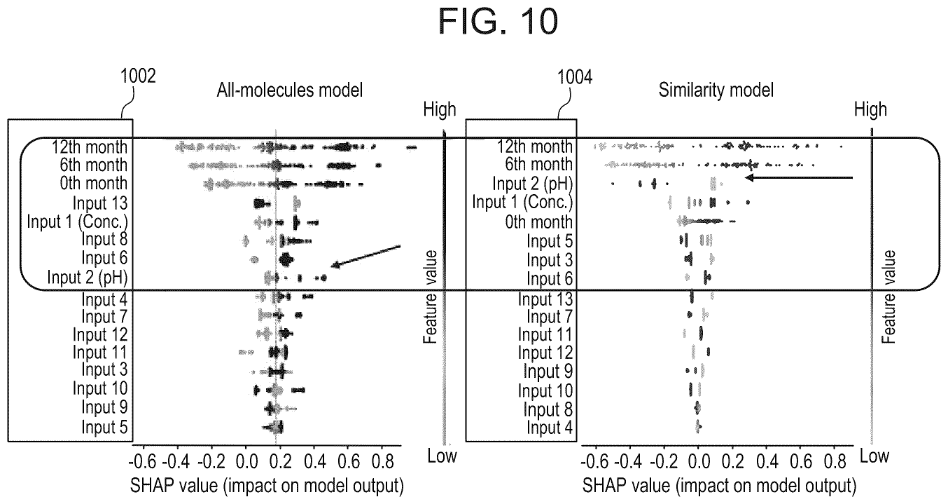

FIG. 10 depicts exemplary diagrams depicting the impact of each training/target characteristic category (or input) on stability predictions. In FIG. 10, the model trained with training characteristic data associated with one or more training pharmaceutical materials (e.g., all molecules model) is shown in plot 1002 and the model trained with training characteristic data associated with a subset of one or more training pharmaceutical materials (e.g., similarity model) is shown in plot 1004. For each plot, the x-axis shows SHAP values, which can be both positive or negative; and the y-axis shows the training/target characteristic categories arranged from higher impact to lower impact from top to bottom. The type of correlation can be depicted by the color of points for each input. From left to right, if the color changes from gray to black, then the training characteristic category can be positive correlated with the predicted stability. On the other hand, if the color changes from black to gray as moving from left to right, then the training characteristic category is negatively correlated with the predicted stability. From both plots 1002 and 1004, the HMW values and concentration are positively correlated with the predicted stability (e.g., the 36-month HMW values). The pH, however, is positively correlated with the predicted stability (e.g., the 36-month HMW values) in plot 1002 and is negatively correlated with the predicted stability (e.g., the 36-month HMW values) in plot 1004.

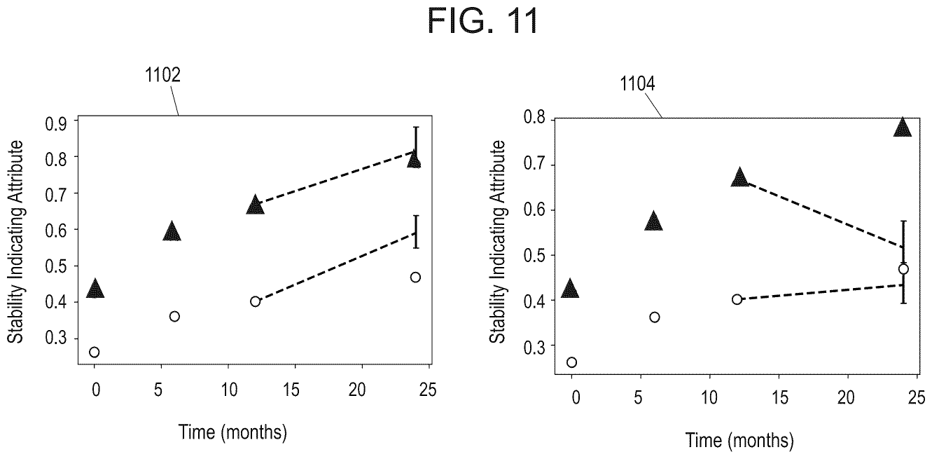

FIG. 11 depicts two exemplary plots 1102 and 1104 showing comparison between the predicted values and measured values for a stability indicating attribute. Plot 1102 shows that the comparison between the predicted values and measured values for a stability indicating attribute for one target pharmaceutical material with two lots (e.g., two samples) by using the second computational model trained with the training characteristic data of the training pharmaceutical materials (e.g., all molecules model data). In plot 1102, the circular points are measured values of stability indicating attribute for lot 1 of target pharmaceutical material at 0 month, 6 months, 12 months, and 24 months; the triangle points are measured values of stability indicating attribute for lot 2 of target pharmaceutical material at 0 month, 6 months, 12 months, and 24 months; the dashed line are predicted values. Plot 1104 shows that the comparison between the predicted values and measured values for a stability indicating attribute for target pharmaceutical material at different lots by using the first computational model trained with the training characteristic data associated with the subset of the one or more training pharmaceutical materials (e.g., similarity model data). In plot 1104, the circular points are measured values of stability indicating attribute for lot 1 of target pharmaceutical material at 0 month, 6 months, 12 months, and 24 months; the triangle points are measured values of stability indicating attribute for lot 2 of target pharmaceutical material at 0 month, 6 months, 12 months, and 24 months; the dashed line are predicted values. According to plots 1102 and 1104, the first computational model demonstrates lower prediction error (or a higher level of prediction accuracy) for predicting values of stability indicating attribute for lot 1 of target pharmaceutical material, and the second computational model demonstrates lower prediction error (or a higher level of prediction accuracy) for predicting values of stability indicating attribute for lot 2 of target pharmaceutical material.

### EXAMPLES

### A. Data Extraction, Data Cleaning and Filtering

The training characteristic data of one or more pharmaceutical materials (e.g., training characteristic data associated with training pharmaceutical materials or prior knowledge data) used for modeling was extracted from one or more data sources. The data sources comprised the values of stability indicating attributes of prior knowledge drug product (or one or more training pharmaceutical materials) or drug substance lots at different temperatures and timepoints along with additional information. One of the data sources was a data table and had information about characteristic data (e.g., molecular properties) of each active ingredient in one or more pharmaceutical materials. To streamline potential variations in naming conventions, a sequence of automated data extraction, cleaning, and filtering steps was adopted. Besides the automated steps, the values of stability indicating attributes of prior knowledge drug product (or one or more training pharmaceutical materials) reported under a different nomenclature (e.g., percentage of aggregate) were also manually added to the original data set. The data cleaning/filtering steps had not altered the numerical values of any entries and the purpose of this procedure was to ensure that related entries were grouped under their respective categories with consistent names (labels). The resulting data set was used for subsequent model training and validation purposes.

### B. Modeling Approaches

After generating the filtered data set, a two-step approach was adopted for developing the predictive models that allowed for leveraging training characteristic data of training pharmaceutical materials up to 24 months. In step 1, the training characteristic data of one or more training pharmaceutical materials (e.g., prior knowledge data of IgG molecules) were used to build a model for predicting 24-month HMW values of the target pharmaceutical material lots stored under RSC. The step 1 was used to assess if the approach and the prior knowledge data employed would result in an accurate and predictive model for predicting stability in 36 months. The performance of the 24-month model (step 1) was assessed by comparing the predicted and actual 24-month HMW values of training pharmaceutical materials. In this step, several machine learning algorithms, including Random Forest (RF), Support Vector Regression (SVR), Partial Least Squares (PLS), Multilayer Perceptron (MLP), and Neural Networks (NN), were evaluated to determine the algorithm that accurately learned the changes in HMW levels over time of the molecules. Then, prior knowledge data from training pharmaceutical materials were used, in step 2, to build the actual model for predicting HMW levels of the target pharmaceutical material after 36 months of storage at RSC. Similar to step 1, the performance of this model was also evaluated independently with training pharmaceutical materials (e.g., mAb3).

### C. Model Training, Tuning, and Validation

For model training and tuning, the leave-one-product-out (LOPO), a version of the k-fold cross-validation scheme, was adopted to identify the hyperparameters for the different machine learning models. LOPO is an iterative approach for the situation when data sets are small, for obtaining a reliable and an unbiased estimate of the model performance. Here, in each iteration, the model was trained using N-1 training pharmaceutical materials, where N was the total number of training pharmaceutical materials, leaving out the data of one of the training pharmaceutical materials for validation. In this situation, models were generalized and were not over-fitted to any specific training pharmaceutical materials. Furthermore, one training pharmaceutical material (mAb3) was completely left out from the LOPO process to serve as an independent test data.

The set of hyperparameters for each AI/ML algorithm used in the LOPO approach is summarized in Table 1. An iterative procedure was adopted, in which a set of values for each hyperparameter was assumed to start with, and sequentially considered for each iteration; for example, assumed values for hyperparameter A: x1, x2, x3, assumed values for hyperparameter B: y1, y2, y3, iteration 1 of LOPO: (x1, y1), iteration 2 of LOPO: (x1, y2), iteration 3 of LOPO: (x1, y3) and so on. The LOPO routine, discussed above, was subsequently employed to build N different models corresponding to the given set of hyperparameters. These models were validated with their respective validation set by calculating the percent error based on the predicted versus the actual measured values. To assign a performance metric for the given hyperparameters, the percent error from each of the N models was averaged and their standard deviation was calculated. This procedure was repeated for all possible combinations of the hyperparameters from the initial sequence. The combination with the lowest mean percent error and standard deviation was considered as the hyperparameter for our model. Finally, with the hyperparameters, the model was retrained on the complete training set (e.g., training characteristic data of training pharmaceutical materials) and predictions were made for the test data set (e.g., target characteristic data of the target pharmaceutical material) and compared against its actual values. One target characteristic data of the target pharmaceutical material (e.g., mAb3) was not used in any of the LOPO iteration steps and thus, is an independent data set for model testing.

TABLE 1
Hyperparameters optimized in different
machine learning (ML) algorithms.

| ML algorithm | Hyperparameters Optimized |
|---|---|
| Random Forest | max. depth, max. features, number |
| | of estimators, min. |
| | samples leaf, min. samples split |
| Support Vector Regression | C, kernel, gamma |
| Partial Least Square | n_components, max iteration |
| Regression | |
| Multilayer Perceptron | activation function, size of hidden |
| | layers 1, 2, and 3, batch size, |
| | learning rate, and early stopping criteria |
| Neural Networks | activation function, size of hidden |
| | layers 1, 2, and 3, batch size, |
| | learning rate, early stopping |
| | criteria, and loss function |

### D. Model Predictions

To make predictions for stability of target pharmaceutical material, each machine learning model was systematically trained P times (P=50) using its hyperparameter in an iterative manner. In each iteration, 15% of the total data set (N, training characteristic data associated with one or more training pharmaceutical materials) was randomly selected as a validation set (Nv) with the remaining being used for model training (N-Nv). To estimate the accuracy of the resulting model, the percent error was calculated by comparing the predicted against the actual values of each data point in both the training and validation sets. This procedure, as mentioned above, was repeated P times resulting in P models with different model parameters, and P different error values for each data point in the total data set (N). These different model parameters were due to variations in the data points in the training and validation data sets, which were selected randomly by the algorithm itself without any human intervention. This way, potential possibilities of error in the prediction due to different model parameters were accounted for in the calculations. Finally, the prediction for each data point in the testing data set (e.g., target characteristic data of the target pharmaceutical material) was obtained by averaging the respective values from the P models. Furthermore, 95% confidence intervals on the predictions were estimated from the percent errors obtained from all the iterations using a t-distribution.

### F. Results and Discussion

In the initial stage of model development, different machine learning algorithms like Random Forest (RF), Support Vector Regression (SVR), Partial Least Squares (PLS), Multilayer Perceptron (MLP), and Neural Networks (NN) were built and tested on the training data set to identify the method that learns the relationship between the input characteristic categories and the HMW values.

Step 1 (e.g., data set and model validation using 24-month HMW values) was to verify if the adopted modeling approach and the prior knowledge data set (e.g., training characteristic data of training pharmaceutical materials) would result in an accurate model for predicting 36-month HMW values for the target pharmaceutical material. A model for predicting 24-month HMW values of target pharmaceutical material was built adopting the approaches discussed earlier (e.g., leave one out) and prior knowledge data set. The results based on generalized error showed that the model predictions for the training data set were in good agreement with their measured values. Also, a lower mAb3 holdout set error indicated that the model could generalize well to molecules that were not part of the training data set. In case of the target pharmaceutical material, the model predictions were in reasonable agreement with the available measured values and these results together confirm that the adopted approach and the prior knowledge data set are suitable for 36-month HMW predictions. The performance of the model was assessed by computing three different percent error metric based on the predicted and experimentally measured values: the generalized error, the target pharmaceutical material error, and mAb3 holdout error. The generalized error, the target pharmaceutical material error, and mAb3 holdout error, which measured the performance of the model on the training characteristic data, the target characteristic data, and mAb3 lots, respectively, are ˜7%, ˜18%, and ˜8%. These values show that, on average, the 24-month model predictions are in reasonable agreement with the measured values.

For the step 1 model validation, input features (e.g., training characteristic categories)—the earlier timepoints with measured HMW values and other common inputs-of the 24-month model were used. Among the various timepoints (0, 3, 6, 9, 12, 18 months) with measured HMW values, 0, 6, and 12 months were chosen as inputs for the predictive 24-month model. Many target pharmaceutical material lots have their HMW values reported at these time points and thus, by choosing them as input parameters, 24-month predictions were made for those target pharmaceutical material lots. Lastly, the predicted values were compared against the measured 24-month values of the respective the target pharmaceutical material lots and the model performance was assessed by computing the average of the percent error. In addition, an independent holdout set comprising mAb3 lots was considered, and the model was used to predict their 24-month HMW values. Akin to the target pharmaceutical material, the predicted values were compared with their measured values and the model performance was determined by calculating the error metric mentioned above. The independent validation set comprising mAb3 lots helped to evaluate the performance of the model and assess its predictive capabilities to a general data set.

Besides evaluating the performance based on the predicted and actual values of the target pharmaceutical material, the model was also independently assessed using mAb3 as a holdout set and the corresponding predictions. Based on the results, the predicted values were close to that of the actual values, except for two lots with the highest HMW values, indicating that the model generalized well to a completely new data set. Based on the error metrics, the generalized error, which denoted the average error in the HMW predictions for the training data set during the leave-one-product-out (LOPO) procedure, was ˜7% for the 24-month model showing that the predictions are in good agreement with the measured values. The target pharmaceutical material error, which denoted the percent error between the predicted 24-month values and the actual values of the target pharmaceutical material lots, was ˜ 18% indicating that the predicted values for the target pharmaceutical material lots were in reasonable agreement with the experimentally measured values. Analogous to the target pharmaceutical material error, the mAb3 holdout error also denoted the percent error between the predicted 24-month and the actual HMW values of mAb3 lots and was ˜8% demonstrating that the 24-month model generalized well to new data set.

In step 2 of model predictions for 36-month HMW values, a model was built for predicting the 36-month HMW values for the target pharmaceutical material lots. For this purpose, a model utilizing HMW values at 0, 6, 12, 24 months as inputs was constructed. Furthermore, mAb3 was again used as an independent holdout data set to deduce the performance of the model. A model for predicting the 36-month HMW values for target pharmaceutical material lots was built. In conclusion, the model predictions and findings showed that the methods to reliably predict HMW values as a function of time for the target pharmaceutical material.

In addition to model predictions, the impact of each input feature (e.g., characteristic category) on the predicted 36-month % HMW value was elucidated using the SHapley Additive explanations (SHAP) technique, as shown in FIGS. 9 and 10. From the resulting feature importance plot, among all the input features, the HMW values at earlier timepoints played a dominant role in determining the HMW value at 36 months. Specifically, the prior HMW values are positively correlated with 36-month predictions and the extent of correlation systematically decreased from time t=24 months to t=0 months. This decreasing correlation indicated that the molecular processes underlying the HMW species formation for the target pharmaceutical material drug product were slowly attaining equilibrium. Besides the earlier timepoint HMW values, other input features such as the protein concentration, glycosylation state, extinction coefficient, average molecular weight, isoelectric point, and chain length also had a marginal impact on the 36-month HMW values.

Analogous to the previous step, the model performance and its predictive capability were assessed using the mAb3 holdout data set. The performance, measured using the average percent error, was about 9% showing that the model predicted lots with lower HMW values accurately but tended to under-predict lots with higher HMW values. The generalized error and the mAb3 holdout data set errors for the model are ˜5% and ˜9%, respectively, showing that the model predicts the HMW values of the training data set and the mAb3 holdout data set equally well. Therefore, these values support the application of the machine learning model for predicting the 36-month HMW values of the target pharmaceutical materials.

An illustrative implementation of a computer system 1200 that may be used in connection with any of the embodiments of the technology described herein (e.g., such as the methods of FIG. 2) is shown in FIG. 12. The computer system 1200 includes one or more processors 1210 and one or more articles of manufacture that comprise non-transitory computer-readable storage media (e.g., memory 1220 and one or more non-volatile storage media 1230). The processor 1210 may control writing data to and reading data from the memory 1220 and the non-volatile storage device 1230 in any suitable manner, as the aspects of the technology described herein are not limited to any particular techniques for writing or reading data. To perform any of the functionality described herein, the processor 1210 may execute one or more processor-executable instructions stored in one or more non-transitory computer-readable storage media (e.g., the memory 1220), which may serve as non-transitory computer-readable storage media storing processor-executable instructions for execution by the processor 1210.

Computer device 1200 may also include a network input/output (I/O) interface 1240 via which the computing device may communicate with other computing devices (e.g., over a network), and may also include one or more user I/O interfaces 1250, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within +20% of a target value in some embodiments, within +10% of a target value in some embodiments, within +5% of a target value in some embodiments, within +2% of a target value in some embodiments. The terms “approximately,” “substantially,” and “about” may include the target value.

1. A computer-implemented method for determining stability of one or more target pharmaceutical materials, the method comprising:
(a) obtaining, via one or more processors, training characteristic data associated with one or more training pharmaceutical materials at one or more training timepoints, wherein the one or more training timepoints comprise at least a first predetermined timepoint;
(b) obtaining, via the one or more processors, target characteristic data associated with the one or more target pharmaceutical materials at one or more target timepoints, wherein the one or more target timepoints are earlier than the first predetermined timepoint;
(c) generating, via the one or more processors, one or more clusters of the one or more training pharmaceutical materials and the one or more target pharmaceutical materials based on the training characteristic data and the target characteristic data;
(d) selecting, via the one or more processors, a subset of the one or more training pharmaceutical materials based on the one or more clusters;
(e) training, via the one or more processors, a first computational model using the training characteristic data associated with the subset of the one or more training pharmaceutical materials at the one or more training timepoints; and
(f) determining, via the one or more processors, the stability of the one or more target pharmaceutical materials at the first predetermined timepoint based on the target characteristic data associated with the one or more target pharmaceutical materials using the trained first computational model.

2. The computer-implemented method of claim 1, wherein the training characteristic data comprise one or more training characteristic categories, and wherein the one or more training characteristic categories comprise at least one of a high molecular weight value, a concentration, or a pH value.

3. The computer-implemented method of claim 1, wherein the target characteristic data comprise one or more target characteristic categories, and wherein the one or more target characteristic categories comprise at least one of a high molecular weight value, a concentration, or a pH value.

4. The computer-implemented method of claim 1, wherein generating the one or more clusters of the one or more training pharmaceutical materials and the one or more target pharmaceutical materials comprises generating the one or more clusters using at least one of a dimensionality reduction technique or a clustering technique.

5. The computer-implemented method of claim 2, further comprising ranking the one or more training characteristic categories based on a level of impact on the determined stability.

6. The computer-implemented method of claim 1, wherein the one or more training pharmaceutical materials are different from the one or more target pharmaceutical materials.

7. The computer-implemented method of claim 1, wherein the one or more training timepoints further comprise a second predetermined timepoint, and wherein the first predetermined timepoint is earlier than the second predetermined timepoint.

8. The computer-implemented method of claim 7, further comprising determining the stability of the one or more target pharmaceutical materials at the second predetermined timepoint based on the target characteristic data associated with the one or more target pharmaceutical materials using the trained first computational model.

9. The computer-implemented method of claim 1, further comprising causing a display to present a visual indication of the determined stability.

10. The computer-implemented method of claim 1, further comprising generating one or more recommendations to adjust at least one storage status associated with the one or more target pharmaceutical materials based on the determined stability.

11. The computer-implemented method of claim 10, further comprising adjusting the at least one storage status associated with the one or more target pharmaceutical materials based on the one or more recommendations.

12. The computer-implemented method of claim 1, further comprising:
training a second computational model using the training characteristic data associated with the one or more training pharmaceutical materials at the one or more training timepoints;
comparing the trained first computational model and the trained second computational model based on a level of prediction accuracy; and
determining the stability of the one or more target pharmaceutical materials at the first predetermined timepoint based on the comparison between the trained first computational model and the trained second computational model and the target characteristic data associated with the one or more target pharmaceutical materials.

13. A computer system for determining stability of one or more target pharmaceutical materials, comprising:
a memory storing instructions; and
one or more processors configured to execute the instructions to perform operations including:
(a) obtaining training characteristic data associated with one or more training pharmaceutical materials at one or more training timepoints, wherein the one or more training timepoints comprise at least a first predetermined timepoint;
(b) obtaining target characteristic data associated with the one or more target pharmaceutical materials at one or more target timepoints, wherein the one or more target timepoints are earlier than the first predetermined timepoint;
(c) generating one or more clusters of the one or more training pharmaceutical materials and the one or more target pharmaceutical materials based on the training characteristic data and the target characteristic data;
(d) selecting a subset of the one or more training pharmaceutical materials based on the one or more clusters;
(e) training a first computational model using the training characteristic data associated with the subset of the one or more training pharmaceutical materials at the one or more training timepoints; and
(f) determining the stability of the one or more target pharmaceutical materials at the first predetermined timepoint based on the target characteristic data associated with the one or more target pharmaceutical materials using the trained first computational model.

14-15. (canceled)

16. The computer system of claim 13, wherein generating the one or more clusters of the one or more training pharmaceutical materials and the one or more target pharmaceutical materials comprises generating the one or more clusters using at least one of a dimensionality reduction technique or a clustering technique.

17-18. (canceled)

19. The computer system of claim 13, wherein the one or more training timepoints further comprise a second predetermined timepoint, and wherein the first predetermined timepoint is earlier than the second predetermined timepoint.

20. The computer system of claim 19, wherein the operations further comprise determining the stability of the one or more target pharmaceutical materials at the second predetermined timepoint based on the target characteristic data associated with the one or more target pharmaceutical materials using the trained first computational model.

21. The computer system of claim 13, wherein the operations further comprise causing a display to present a visual indication of the determined stability.

22. The computer system of claim 13, wherein the operations further comprise generating one or more recommendations to adjust at least one storage status associated with the one or more target pharmaceutical materials based on the determined stability.

23. (canceled)

24. The computer system of claim 13, wherein the operations further comprise:
training a second computational model using the training characteristic data associated with the one or more training pharmaceutical materials at the one or more training timepoints;
comparing the trained first computational model and the trained second computational model based on a level of prediction accuracy; and
determining the stability of the one or more target pharmaceutical materials at the first predetermined timepoint based on the comparison between the trained first computational model and the trained second computational model and the target characteristic data associated with the one or more target pharmaceutical materials.

25. A non-transitory computer-readable medium containing instructions for determining stability of one or more target pharmaceutical materials that, when executed by a processor, cause the processor to perform a method comprising:
(a) obtaining training characteristic data associated with one or more training pharmaceutical materials at one or more training timepoints, wherein the one or more training timepoints comprise at least a first predetermined timepoint;
(b) obtaining target characteristic data associated with the one or more target pharmaceutical materials at one or more target timepoints, wherein the one or more target timepoints are earlier than the first predetermined timepoint;
(c) generating one or more clusters of the one or more training pharmaceutical materials and the one or more target pharmaceutical materials based on the training characteristic data and the target characteristic data;
(d) selecting a subset of the one or more training pharmaceutical materials based on the one or more clusters;
(e) training a first computational model using the training characteristic data associated with the subset of the one or more training pharmaceutical materials at the one or more training timepoints; and
(f) determining the stability of the one or more target pharmaceutical materials at the first predetermined timepoint based on the target characteristic data associated with the one or more target pharmaceutical materials using the trained first computational model.

26-36. (canceled)
